SEQUENCE LISTING (11 sequences)

SEQ ID NO: 1 - DNA, 50 bp, synthetic construct
  misc_feature 1..50: note = synthetic oligonucleotide
  source 1..50: mol_type = other DNA
atgggtggacgtgacactatagcagcatgtcaagatcacagattttgggc

SEQ ID NO: 2 - DNA, 55 bp, synthetic construct
  misc_feature 1..55: note = synthetic oligonucleotide
  source 1..55: mol_type = other DNA
atgggtggactttaggtgacactatagcagcatgtcaagatcacagattttgggc

SEQ ID NO: 3 - DNA, 60 bp, synthetic construct
  misc_feature 1..60: note = synthetic oligonucleotide
  source 1..60: mol_type = other DNA
atgggtggacagctatttaggtgacactatagcagcatgtcaagatcacagattttgggc

SEQ ID NO: 4 - DNA, 65 bp, synthetic construct
  misc_feature 1..65: note = synthetic oligonucleotide
  source 1..65: mol_type = other DNA
atgggtggacagtcaagctatttaggtgacactatagcagcatgtcaagatcacagattttgggc

SEQ ID NO: 5 - DNA, 70 bp, synthetic construct
  misc_feature 1..70: note = synthetic oligonucleotide
  source 1..70: mol_type = other DNA
atgggtggaccggccagtcaagctatttaggtgacactatagcagcatgtcaagatcacagattttgggc

SEQ ID NO: 6 - DNA, 75 bp, synthetic construct
  misc_feature 1..75: note = synthetic oligonucleotide
  source 1..75: mol_type = other DNA
atgggtggacaacgacggccagtcaagctatttaggtgacactatagcagcatgtcaagatcacagattttgggc

SEQ ID NO: 7 - DNA, 80 bp, synthetic construct
  misc_feature 1..80: note = synthetic oligonucleotide
  source 1..80: mol_type = other DNA
atgggtggaccgtaaaacgacggccagtcaagctatttaggtgacactatagcagcatgtcaagatcacagattttgggc

SEQ ID NO: 8 - DNA, 85 bp, synthetic construct
  misc_feature 1..85: note = synthetic oligonucleotide
  source 1..85: mol_type = other DNA
atgggtggactatgacgtaaaacgacggccagtcaagctatttaggtgacactatagcagcatgtcaagatcacagattttgggc

SEQ ID NO: 9 - DNA, 90 bp, synthetic construct
  misc_feature 1..90: note = synthetic oligonucleotide
  source 1..90: mol_type = other DNA
atgggtggacacagctatgacgtaaaacgacggccagtcaagctatttaggtgacactatagcagcatgtcaagatcacagattttgggc

SEQ ID NO: 10 - DNA, 95 bp, synthetic construct
  misc_feature 1..95: note = synthetic oligonucleotide
  source 1..95: mol_type = other DNA
atgggtggacaggaaacagctatgacgtaaaacgacggccagtcaagctatttaggtgacactatagcagcatgtcaagatcacagattttgggc

SEQ ID NO: 11 - DNA, 100 bp, synthetic construct
  misc_feature 1..100: note = synthetic oligonucleotide
  source 1..100: mol_type = other DNA
atgggtggactctccaggaaacagctatgacgtaaaacgacggccagtcaagctatttaggtgacactatagcagcatgtcaagatcacagattttgggc

(19) United States
(12) Patent Application Publication
(10) Pub. No.: US 2026/0234710 A1
(43) Pub. Date: Aug. 13, 2026

(54) GENE ANALYSIS METHOD, GENE ANALYSIS DEVICE, AND GENE ANALYSIS KIT

(71) Applicant: HITACHI, LTD., Tokyo (JP)

(72) Inventors: Takahiro Ando, Tokyo (JP); Takahide Yokoi, Tokyo (JP); Takashi Anazawa, Tokyo (JP); Chihiro Manri, Tokyo (JP); Shoko Kawakami, Tokyo (JP); Takeshi Ishida, Tokyo (JP)

(73) Assignee: HITACHI, LTD., Tokyo (JP)

(21) Appl. No.: 19/147,897

(22) PCT Filed: Jan. 18, 2024
(86) PCT No.: PCT/JP2024/001209

§ 371 Date: Jul. 14, 2025

(30) Foreign Application Priority Data
Apr. 3, 2023 (JP) 2023-060234

(51) Int. Cl. C12Q 1/6855; C12Q 1/6827; C12Q 1/686; C12Q 1/6876

(52) U.S. Cl. CPC C12Q 1/6855; C12Q 1/6827; C12Q 1/686; C12Q 1/6876; C12Q 2600/156

(57) ABSTRACT

The invention relates to a gene mutation analysis method, and a gene analysis device and a gene analysis kit based on the method. Specifically, the invention relates to a gene analysis method for detecting a target base sequence. The gene analysis method includes: a step of performing a base extension reaction using a test nucleic acid as a template by using a primer specific for the target base sequence; a step of subjecting a product of the reaction to electrophoresis or mass spectrometry; and a step of detecting a wild type and a mutant of the target base sequence in the test nucleic acid based on a difference in size of the product determined by the electrophoresis or the mass spectrometry and information on the wild type and the mutant of the target base sequence. The step of performing the base extension reaction includes performing the base extension reaction using two or more types of primers with different base lengths for a same target base sequence.

### TECHNICAL FIELD

The present invention relates to a gene mutation analysis method, and a gene analysis device and a gene analysis kit based on the method.

### BACKGROUND ART

With recent advances in genome analysis technique, an association between various diseases and gene mutations is becoming clearer. Somatic mutations, which are acquired gene mutations resulting from diseases such as cancer, have a characteristic that a mutation occurrence site on the genome cannot be predicted and a mutant allele frequency within an individual or tissue cannot be predicted. For example, cancer cells and normal cells are contained in a tissue sample from a tumor portion excised from a cancer patient, and a gene mutation is diversified even between cancer cells. Therefore, a proportion of cells harboring a gene mutation at a specific site of a specific gene within a sample may be extremely low. Therefore, a highly sensitive detection method has been required to detect an acquired gene mutation resulting from a disease. When selecting a therapeutic method or a therapeutic agent, not only the presence or absence of a gene mutation at a specific site of a target gene but also a mutant allele frequency thereof may be used as an index. Therefore, in addition to highly sensitive detection of gene mutations, quantification of the mutant allele frequency thereof is also important.

In searching for disease-related gene mutations that can serve as biomarkers, next generation sequencers (NGS) are now being used to enable large-scale, high-speed analysis, and item extraction of gene mutations is becoming easy. Therefore, in diagnosis of diseases including cancer, there is a trend to increase versatility of a testing flow by measuring a gene mutation identified based on a result of comprehensive analysis by NGS using a gene mutation detection technique that is more advantageous than NGS in terms of cost and detection sensitivity.

Examples of the low-cost gene mutation detection technique include fragment analysis using capillary electrophoresis (CE). Typical examples thereof include single nucleotide polymorphism (SNP) genotyping and a multiplex ligation-dependent probe amplification (MLPA) (registered trademark) assay. Both techniques are established as a technique capable of detecting a gene mutation by fragment analysis by applying a primer to a region of interest of a gene sequence.

NPL 1 shows that by using SNapshot (registered trademark) as a SNP genotyping kit, a target tumor-derived gene sequence in a formalin-fixed and paraffin-embedded tissue can be selectively enriched by a multiplex polymerase chain reaction (PCR), and then 120 known gene mutations in 13 cancer genes can be detected. In measuring the KRAS G12D mutation in the human lung cancer A427 cell line and the EGFR T790M mutation in the lung adenocarcinoma cell line NCI-H1975, it has been shown that a detection sensitivity of a mutant with respect to a wild type is approximately 3%. An allele frequency that can be detected with the SNapshot (registered trademark) kit is typically said to be about 5%.

In recent years, in mutation detection using NGS, a detection sensitivity is achieved at less than 1% by using a molecular barcode, and many technical innovations aimed at increasing a sensitivity in capillary electrophoresis analysis have been reported. For example, PTL 1 discloses, as a data processing technique for increasing a sensitivity and expanding a quantification range (increasing a dynamic range) without constructing a database in advance, a method of selecting specific wavelength data from electrophoresis data, performing a filtering process of cutting a part or all of the specific wavelength data on a high frequency side, comparing peak intensities of the specific wavelength data before and after the filtering process for each cutoff frequency, calculating, as a first cutoff frequency, a lowest cutoff frequency at which a decrease in peak intensity of the specific wavelength data is within a predetermined allowable range, and correcting first data or data after color call for the first data by performing a filtering process using the first cutoff frequency. As an innovation in hardware of a light emission detection device, PTL 2 discloses a method of reducing a size of the light emission detection device and simultaneously realizing a high sensitivity and low crosstalk by individually condensing light emitted from each light emitting point of a light emitting point array by each condenser lens of a condenser lens array to form a light flux, deflecting each light flux by an optical element to form a deflected light flux, causing the deflected light flux to enter a sensor in parallel and detecting the light flux, and satisfying a predetermined relationship among a diameter of each light emitting point, a focal length and an interval of each condenser lens, and an optical path length of each condenser lens and the optical element.

### CITATION LIST

### Patent Literature

PTL 1: JP2022-149047A
PTL 2: JP7075974B

### Non-Patent Literature

NPT 1: Dias-Santagata, D. et al., EMBO Molecular Medicine Vol. 2, pp. 146-158 (2010)

### SUMMARY OF INVENTION

### Technical Problem

As a low-cost gene mutation detection technique, fragment analysis in capillary electrophoresis is exemplified, but a detection sensitivity of a mutant with respect to a wild type is generally considered to be a few percent, and there is a trend toward increasing the sensitivity of the technique in order to demonstrate a clinical value. Further, there is a need not only to determine the presence or absence of a gene mutation, but also to quantify a mutant allele frequency thereof. Generally, a gene mutation is detected by using one type of primer for one type of gene target. However, there is a problem in that it is difficult to quantify a low-level gene mutation that occurs at a low frequency of, for example, less than 1% with a high sensitivity and accuracy using only the current capillary electrophoresis technique.

### Solution to Problem

As a result of intensive studies to solve the above-described problems, it has been found that quantification accuracy of gene mutation detection can be improved by applying a plurality of primers to one type of gene target to increase a coverage of a target gene (a target base sequence). For example, when detecting a gene mutation by combining a single base extension reaction and electrophoresis, two or more types of primers with different base lengths that contain the same base sequence region are used to perform the single base extension reaction on a target gene, and the product is then subjected to electrophoresis for analysis, whereby a variation in value of a mixing ratio of a mutant to a wild type in the target gene can be reduced.

Therefore, in one aspect, the invention relates to a gene analysis method for detecting a target base sequence, wherein the gene analysis method includes: a step of performing a base extension reaction using a test nucleic acid as a template by using a primer specific for the target base sequence; a step of subjecting a product of the reaction to electrophoresis or mass spectrometry; and a step of detecting a wild type and a mutant of the target base sequence in the test nucleic acid based on a difference in size of the product determined by the electrophoresis or the mass spectrometry and information on the wild type and the mutant of the target base sequence, wherein the step of performing the base extension reaction includes performing the base extension reaction using two or more types of primers with different base lengths for a same target base sequence.

In one embodiment, the gene analysis method includes: a step of performing a single base extension reaction using a test nucleic acid as a template by using a single base extension reaction primer for detecting the target base sequence and a single base extension reaction substrate having a fluorescent dye; a step of subjecting a product of the single base extension reaction to electrophoresis; and a step of detecting a wild type and a mutant of the target base sequence in the test nucleic acid based on a difference in size of the product as a mobility of the electrophoresis and information on the wild type and the mutant of the target base sequence as a fluorescence signal of the fluorescent dye, wherein the step of performing the single base extension reaction includes performing the single base extension reaction using two or more types of single base extension reaction primers with different base lengths for a same target base sequence.

In one embodiment, the gene analysis method includes: a step of performing a single base extension reaction using a test nucleic acid as a template by using a single base extension reaction primer for detecting the target base sequence; a step of subjecting a product of the single base extension reaction to mass spectrometry; and a step of detecting a wild type and a mutant of the target base sequence in the test nucleic acid based on a difference in size of the product as a difference in signal of the mass spectrometry and information on the wild type and the mutant of the target base sequence as a difference in signal of the mass spectrometry of a base incorporated in the single base extension reaction, wherein the step of performing the single base extension reaction includes performing the single base extension reaction by using two or more types of single base extension reaction primers with different base lengths for a same target base sequence.

In one embodiment, the gene analysis method includes: a step of performing a multi-base extension reaction using the test nucleic acid as a template by using a multi-base extension reaction primer for detecting the target base sequence and a multi-base extension reaction substrate having a fluorescent dye; a step of subjecting a product of the multi-base extension reaction to electrophoresis; and a step of detecting a wild type and a mutant of the target base sequence in the test nucleic acid based on a difference in size of the product as a mobility of the electrophoresis and information on the wild type and the mutant of the target base sequence as a mobility of the electrophoresis and a fluorescence signal of the fluorescent dye, wherein the step of performing the multi-base extension reaction includes performing the multi-base extension reaction using two or more types of multi-base extension reaction primers with different base lengths for a same target base sequence.

In another aspect, the invention relates to a gene analysis method for detecting a target base sequence, wherein the gene analysis method includes: a step of binding, to a test nucleic acid, a forward primer and a reverse primer which specifically hybridize to the target base sequence; a step of performing an amplification reaction using a primer labeled with a fluorescent dye and using, as a template, a ligated probe obtained by ligating the forward primer and the reverse primer; a step of subjecting a product of the amplification reaction to electrophoresis; and a step of detecting a wild type and a mutant of the target base sequence in the test nucleic acid based on a difference in size of the product as a mobility of the electrophoresis and information on the wild type and the mutant of the target base sequence as a fluorescence signal of the fluorescent dye, wherein the reverse primer includes an inserted base sequence for adjusting a base length, thereby allowing two or more types of reverse primers with different base lengths to bind to the test nucleic acid.

In yet another aspect, the invention relates to a gene analysis device for implementing the gene analysis method, wherein the gene analysis device includes: a measurement unit configured to perform a single base extension reaction, electrophoresis, and measurement of a fluorescence signal; a data analysis unit including a data processing device and a measurement data storage unit that stores measurement data obtained by the measurement unit; and a control unit, wherein the control unit is configured to determine a number, a type, and a base length of two or more types of primers to be used based on a mutation detection percentage of a target base sequence.

In yet another aspect, the invention relates to a gene analysis kit for implementing the gene analysis method, wherein the gene analysis kit includes: a single base extension reaction primer for detecting a target base sequence; and a single base extension reaction substrate having a fluorescent dye, wherein the single base extension reaction primer includes two or more types of primers with different base lengths for a same target base sequence.

The present description includes disclosures of Japanese Patent Application No. 2023-060234 filed on Apr. 3, 2023, based on which the present application claims priority.

### Advantageous Effects of Invention

According to the invention, by applying a plurality of primers to one type of target gene (target base sequence) to increase a coverage of the target gene, quantification accuracy of a gene mutation can be increased, and accuracy of disease diagnosis related to a gene abnormality such as cancer can be increased. Without requiring special processing of electrophoresis data or changes to hardware, data reliability can be improved in quantitative analysis of a low-level gene mutation with a mutant allele frequency of 1% or less by simply modifying a reagent. Problems, configurations, and effects other than those described above will be clarified by the following description of embodiments.

### BRIEF DESCRIPTION OF DRAWINGS

FIG. 1 is a schematic diagram of fragment analysis of capillary electrophoresis for quantitatively measuring a ratio of a mutant to a wild type of a target base sequence using a single base extension reaction.

FIG. 2 is a schematic diagram illustrating an embodiment in which the single base extension reaction is performed by applying a plurality of primers with different base lengths to one type of the same target base sequence.

FIG. 3 is a schematic diagram illustrating an embodiment in which a primer for a target base sequence and a complementary strand primer are used in quantitative measurement of a ratio of a mutant to a wild type of a target base sequence using the single base extension reaction.

FIG. 4 is a schematic diagram illustrating an embodiment of detection of a target base sequence by an MLPA assay.

FIG. 5 is a diagram illustrating a percentage of fluorescence intensity peak ratio corresponding to a separation in an electrophoretic mobility (a base length) when a fluorescence signal of a labeled fluorescent dye that is detected by capillary electrophoresis is assumed to be a Gaussian function.

FIG. 6, A is a diagram illustrating a result of fragment analysis in which the single base extension reaction is performed using 11 types of primers with different base lengths when a ratio of a mutant to a wild type is 1% with a gene mutation EGFR L858R targeted, and FIG. 6, B is a diagram illustrating a result of fragment analysis in which the single base extension reaction is performed using 11 types of primers with different base lengths when the ratio of the mutant to the wild type is changed with the gene mutation EGFR L858R targeted.

FIG. 7 is a flowchart illustrating an example of a processing procedure in a gene analysis device and a gene analysis kit for implementing the invention.

FIG. 8 is a block configuration diagram illustrating an example of functions of the gene analysis device of the invention.

FIG. 9 is a diagram illustrating an example of a user interface screen output by the gene analysis device of the invention.

### DESCRIPTION OF EMBODIMENTS

Hereinafter, an example of the embodiment of the invention will be described with reference to the drawings.

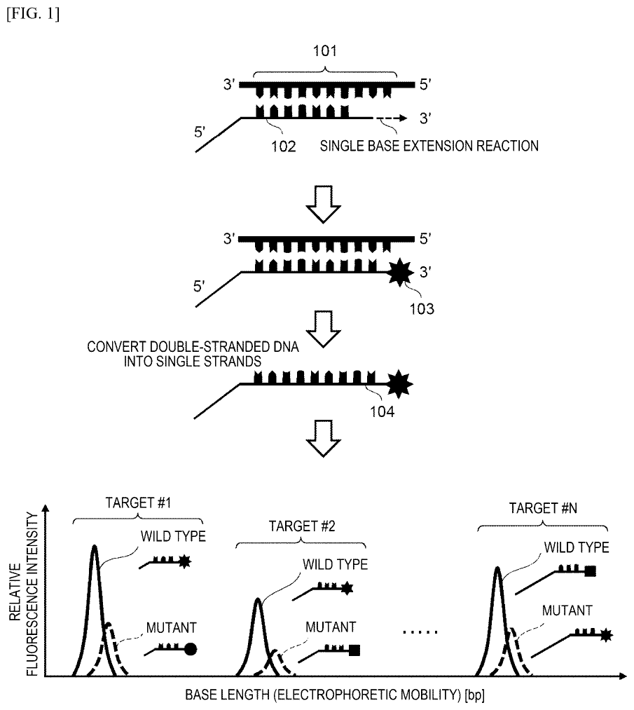

FIG. 1 is a schematic diagram of fragment analysis of capillary electrophoresis for quantitatively measuring a ratio of a mutant to a wild type of a target base sequence using a single base extension reaction. Generally, it is designed such that products obtained by performing the single base extension reaction using a target base sequence 101 as a template and a primer 102 with a different base length (molecular weight) for each target have different electrophoretic mobilities. By a polymerase reaction, ddNTP modified with four colors (four types) of fluorescent dyes 103, whose terminal base corresponds to adenine (A), cytosine (C), guanine (G), and thymine (T), may be added to the position of 3′ end of the primer 102 corresponding to a gene mutation through the single base extension reaction. A double-stranded DNA may be converted into single strands by a formamide treatment and heat denaturation, and a gene mutation may be identified by fluorescent detection of the fluorescent dye at the 3′ end of a product 104 of the single base extension reaction. In SNAPshot (registered trademark), which is a representative gene mutation detection kit using the method, it is also assumed that one type of primer is assigned to one type of target, and primers with different base lengths are assigned to respective targets. Since the primers have different base lengths for each target, an electrophoretic mobility is different, and academic research to date has shown that 20 or more types of known gene mutations can be detected with a round of electrophoresis. At this time, not only a single nucleotide polymorphism (SNP) in which only one base is mutated can be detected, but also a gene sequence having a specific length can be detected by designing a primer. Further, insertion (IN) and deletion (DEL), which are types of gene mutation, can also be detected based on the same principle. This method can also be used to detect a specific germline gene mutation in microsatellite instability (MSI) known as a phenomenon in which a microsatellite repeat sequence shows a different number of repeats in a tumor tissue compared to a normal tissue due to a decrease in ability to repair errors in a base sequence that occur during DNA replication. In addition to the target of gene mutation, similarly, in the MassARRAY (registered trademark) system, which is a method using mass spectrometry without capillary electrophoresis as a method for detecting a single base extension product, one type of primer is assigned to one type of target, and primers with different base lengths are assigned to respective targets, with a maximum number of mutations that can be simultaneously detected in SNP typing being 40.

According to the invention, precision and accuracy of mutation detection in a target base sequence can be improved by designing a plurality of primers for one type of target base sequence. Therefore, in one aspect, the invention relates to a gene analysis method for detecting a target base sequence, wherein the gene analysis method includes: a step of performing a base extension reaction using a test nucleic acid as a template by using a primer specific for the target base sequence; a step of subjecting a product of the reaction to electrophoresis or mass spectrometry; and a step of detecting a wild type and a mutant of the target base sequence in the test nucleic acid based on a difference in size of the product determined by the electrophoresis or the mass spectrometry and information on the wild type and the mutant of the target base sequence, wherein the step of performing the base extension reaction includes performing the base extension reaction using two or more types of primers with different base lengths for a same target base sequence.

According to the invention, the target base sequence refers to a base sequence for which detection (and quantification) is desired. For example, the target base sequence may be a base sequence containing a gene mutation, and the target base sequence may be detected (and quantified) by distinguishing between a wild type and a mutant. Examples of such a gene mutation may include, but are not limited to, a single nucleotide polymorphism (SNP), an insertion (IN) mutation, a deletion (DEL) mutation, a microsatellite repeat sequence, a copy number variation (CNV), and an epigenetic mutation. The mutant of the target base sequence may include one type of mutant or may include a plurality of types of mutants. For example, there is a gene mutation in which a wild type and two or three mutants are present, and such a plurality of mutants can also be distinguished and detected (and quantified) according to the invention.

The test nucleic acid used in the present method may not be particularly limited as long as it is a nucleic acid sample to be detected for the target base sequence, and may include deoxyribonucleic acid (DNA) such as genomic DNA and cDNA, and ribonucleic acid (RNA) such as messenger RNA (mRNA) and a fragment thereof. According to the invention, it may be preferable to use, for example, cell-free DNA (cfDNA, DNA free in the blood) or circulating tumor DNA (ctDNA) as the test nucleic acid. The nucleic acid can be prepared from the sample by a method known in the art. A kit for preparing the nucleic acid is commercially available from many manufacturers, and the target nucleic acid can be easily purified.

Hereinafter, each embodiment of the gene analysis method according to the invention will be described.

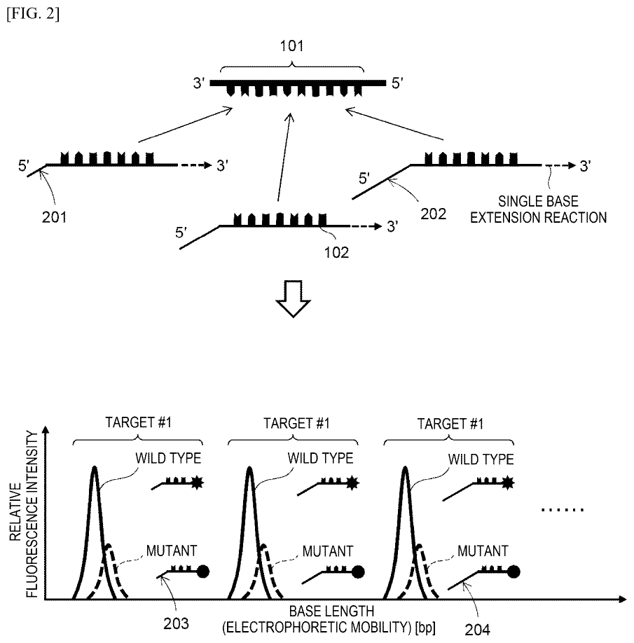

FIG. 2 is a diagram illustrating an embodiment in which a single base extension reaction is performed by applying a plurality of primers with different base lengths to one type of the same target base sequence. As compared with the primer 102 in the above-described general method illustrated in FIG. 1, a primer 201 with a short-adjusted base length and a primer 202 having a long-adjusted base length are applied to one type of the same target base sequence 101 to perform the single base extension reaction. Accordingly, for one type of the same target base sequence, fluorescence signals of products of the single base extension reaction that have different base lengths can be detected. Although the same fluorescent color signal is detected in both a product 203 from the primer 201 with a short-adjusted base length and a product 204 from the primer 202 with a long-adjusted base length, base lengths (sizes) may be different, resulting in different electrophoretic mobilities and different timings at which the fluorescence signals are detected. Therefore, a coverage of the same target base sequence (target #1 in FIG. 2) may be increased, the number of times of detection may be increased by the number of primers, and thus reliability of a gene mutation detection result may be increased. That is, it is expected that the variability may be reduced and precision may be increased while at the same time improving accuracy.

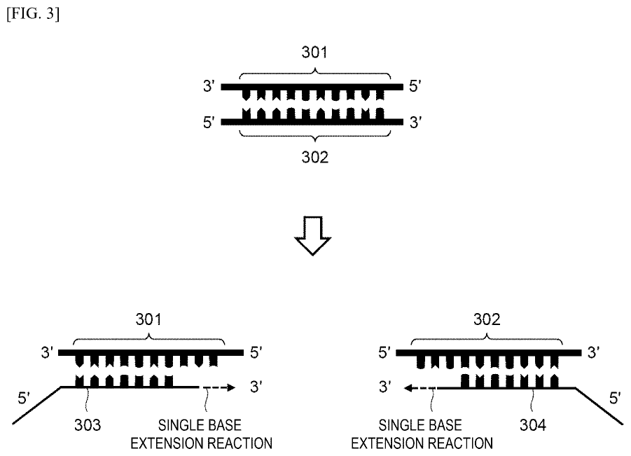

FIG. 3 is a diagram illustrating an embodiment in which a primer for a target base sequence and a complementary strand primer are used in quantitative measurement of a ratio of a mutant to a wild type of a target base sequence using the single base extension reaction. Similar to the procedure illustrated in FIG. 1, a target base sequence (a template) 301 may be used as a template, and a complementary sequence (a template antisense) 302 of the target base sequence may be used as a template. When the target base sequence 301 is used as a template, a primer (a forward primer) 303 with an adjusted base length can be used, and when the complementary sequence (the template antisense) 302 of the target base sequence is used as a template, a complementary strand primer (a reverse primer) 304 with an adjusted base length can be used to perform the single base extension reaction. Accordingly, when the accuracy or sensitivity of detection of the target base sequence is insufficient with only the forward primer, the quantification accuracy of the gene mutation can be improved by applying a plurality of primers with different base lengths to one type of the same target base sequence using the reverse primer.

Therefore, in one embodiment, the method of the invention includes: a step of performing a single base extension reaction using a test nucleic acid as a template by using a single base extension reaction primer for detecting the target base sequence and a single base extension reaction substrate having a fluorescent dye; a step of subjecting a product of the single base extension reaction to electrophoresis; and a step of detecting a wild type and a mutant of the target base sequence in the test nucleic acid based on a difference in size of the product as a mobility in the electrophoresis and information on the wild type and the mutant of the target base sequence as a fluorescence signal of the fluorescent dye, wherein the step of performing the single base extension reaction includes performing the single base extension reaction using two or more types of single base extension reaction primers with different base lengths for a same target base sequence.

The method of the embodiment is based on a gene analysis method by a combination of the single base extension reaction and electrophoresis, and such a gene analysis method is well known in the art, for example, as described in NPL 1.

The single base extension reaction may be performed using a single base extension reaction primer for detecting a target base sequence in the presence of a substrate (dideoxybase triphosphate: ddNTP) to which a fluorescent dye is bound, and according to the present invention, two or more types of primers with different base lengths may be used for the same target base sequence as the single base extension reaction primer.

The single base extension reaction primer may be either DNA or RNA, and may be selected according to the types of a test sample and a target base sequence, and the type of a polymerase to be used in the single base extension reaction. Preferably, the primer may be DNA, and the single base extension reaction may be performed using DNA or mRNA as a template as a test nucleic acid.

According to the invention, the single base extension reaction primer may include two or more types of primers with different base lengths for the same target base sequence. For example, 2 to 20 types, preferably 2 to 12 types of primers with different base lengths may be included. Here, the expression “with different base lengths” means that a plurality of products obtained using the primers are different in base length to such an extent that they can be distinguished from each other based on a size (a base length) thereof. A specific difference in base length may vary depending on a detection method (electrophoresis, mass spectrometry, or the like), and may be set to, for example, 3 bp or more, preferably 4 bp or more, or 5 bp or more.

The single base extension reaction primer may be primers having the same orientation or primers having different orientations as long as the primers have different base lengths. For example, the two types of primers may be the same forward primer, or may be a combination of a forward primer and a reverse primer.

When detecting a plurality of (for example, two or more types of) target base sequences, two or more types of primers with different base lengths may be used for each target base sequence. Since the primer is necessary for each target base sequence, the number of primers can be designed according to the type of the target base sequence to be detected. For example, in order to detect five or more types (for example, 5 to 100 types), preferably 10 or more types (for example, 10 to 100 types) of target base sequences, the primers include two or more types of primers with different base lengths for each target base sequence.

Each primer is designed to have a sequence specific for the target base sequence, that is, a sequence complementary to the target base sequence. The target base sequence to be detected may be selected based on a base sequence of the primer. The base length of the primer affects the base length (the size) of the product of the single base extension reaction, and thus a mobility of electrophoresis may be decided based on the base length of the primer. According to the present invention, it may be necessary to appropriately design the base sequence and base length of the primer.

A design method of the primer is well known in the art, and the primer that can be used in the invention may be designed to satisfy conditions that allow specific annealing, for example, to have a length and a base composition (a melting temperature) that allow specific annealing. For example, the length that functions as a primer may preferably be 10 bases or more, more preferably 15 bases to 50 bases, still more preferably 15 bases to 30 bases, for example, about 20 bases. In designing, it may be preferable to confirm a GC content of the primer and a melting temperature (Tm) of the primer. For confirmation of Tm, known primer design software can be used. The designed primer can be chemically synthesized by a known oligonucleotide synthesis method, and is generally synthesized using a commercially available chemical synthesis device.

The primers may have different base lengths by being provided with tags (molecular weights) for adjusting an electrophoretic mobility, for example, interstrand-crosslinked double-stranded DNA tags. The inventors previously developed a fragment analysis method using capillary electrophoresis, and developed an analysis method capable of expanding the number of gene mutations that can be simultaneously detected to several tens to several hundreds. Specifically, by linking and using an interstrand-crosslinked double-stranded DNA tag to a primer, a base length of the primer can be extended by changing a length of the double-stranded DNA tag, and an electrophoretic distance can be stably extended to 120 bp or more, and the number of gene mutations that can be detected simultaneously can be increased. The double-stranded DNA tags may have distinguishable lengths based on a mobility thereof and contain at least one interstrand crosslink. According to the present invention, the term “interstrand crosslink” means that one strand and the other strand in the double-stranded DNA are crosslinked at least at one site. Such a method for intramolecularly cross-linking two strands is not particularly limited as long as it is a method known in the art. Preferably, the interstrand crosslink may be formed via photo-crosslinking. The double-stranded DNA tag containing the interstrand crosslink defines a migration distance (a mobility) in electrophoresis. That is, the migration distance during the electrophoresis can be changed by linking double-stranded DNA tags with different lengths to the primers. In the capillary electrophoresis, a nucleic acid with a base length of up to about 600 bases can be detected, so that the length of the double-stranded DNA tag can range from 1 base to about 590 bases, excluding a base length (10 bases to 30 bases) of a portion of the primer that binds to the target base sequence. The base sequence of the double-stranded DNA tag may not be particularly limited as long as the base sequence is a nucleic acid having an interstrand crosslink. The double-stranded DNA tag can be chemically synthesized by a known oligonucleotide synthesis method, and is generally synthesized using a commercially available chemical synthesis device.

At this time, a plurality of primers with different base lengths may be used, and when a target base sequence for which quantitative gene analysis is desired is included, it may be advisable to place a target base sequence item for which higher quantitation is required in a base length region where resolution and accuracy of the capillary electrophoresis device is high (generally set to around 50 bp to 100 bp, but varying depending on the device specification). Due to the nature of fragment analysis, the resolution of electrophoresis tends to decrease as the base length increases, and therefore the detection accuracy can be improved by using a short base primer for the target base sequence for which higher accurate quantitation is required. Therefore, in one embodiment, when detecting a plurality of target base sequences, the single base extension reaction primer for detecting a target base sequence for which quantification is desired is designed to have a base length shorter than that of the single base extension reaction primer for detecting another target base sequence.

In the embodiment of the method of the invention, the single base extension reaction may be performed using the above-described primer in the presence of the single base extension reaction substrate having a fluorescent dye. The single base extension reaction is known in the art, and is typically a single base extension reaction using a polymerase. The polymerase to be used may be selected according to the type of template (test sample) and the type of primer to be used. For example, a DNA-dependent or RNA-dependent DNA polymerase may be used for the single base extension reaction with a DNA primer using DNA or RNA as a template, respectively.

The single base extension reaction is widely known in the art, and for example, NPL 1 or the like describes a method of efficiently extending one base by a cycle reaction.

When a target base sequence is present, a primer may hybridize to the target base sequence, and a nucleotide may be incorporated as a substrate at the 3′ end portion of the primer by a synthetic reaction of polymerase. In this case, by using, for example, a dideoxynucleotide (ddNTP) as the nucleotide (the substrate) to be incorporated, the synthesis reaction may be completed with only one base extension.

According to the invention, a substrate having a fluorescent dye may be used as a substrate for the single base extension reaction, and the substrate may contain two or more types of substrates having different fluorescent dyes. The fluorescent dye is useful for easily detecting whether the substrate is incorporated or for determining the type of the incorporated base, and a fluorescent dye known in the art can be used. Examples of the fluorescent dye may include, but are not limited to, fluorescein, fluorescein isothiocyanate (FITC), sulforhodamine (TR), tetramethylrhodamine (TRITC), carboxy-X-rhodamine (ROX), carboxytetramethylrhodamine (TAMRA), NED, 5-carboxyfluorescein (5-FAM), 6-carboxyfluorescein (6-FAM), 5′-hexachlorofluorescein CE-phosphoramidite (HEX), 6-carboxy-4′,5′-dichloro-2′,7′-dimethoxyfluorescein (JOE), 5′-tetrachlorofluorescein CE-phosphoramidite (TET), rhodamine 110 (R110), rhodamine 6G (R6G), VIC (registered trademark), ATTO series, Alexa Fluor (registered trademark) series, Texas red, and Cy series, and examples of the fluorescent dye that does not cause a shift in electrophoretic size may include dR110 (carboxy-dichloro rhodamine 110), dR6G (dihydro rhodamine 6G), dTAMRA (tetramethyl rhodamine), and dROX (carboxy-X-rhodamine). For example, when trying to determine the type of base, five types of fluorescent dyes that are excited and detected at different wavelengths can be used in combination to identify five types including four types of bases and a reference (to detect and correct a base length from a reference ladder DNA). The type, the introduction method, and the like of such a fluorescent dye may not be particularly limited, and various known method in the related art can be used.

After the single base extension reaction, the obtained product may be subjected to electrophoresis and analyzed. The electrophoresis may not be particularly limited as long as it is a measurement method capable of performing fragment analysis by electrophoresis, and for example, electrophoresis in a microchannel such as capillary electrophoresis (CE) or micro-electro-mechanical systems (MEMS) can be used. In a preferred embodiment, the electrophoresis is capillary electrophoresis (CE).

The electrophoresis, for example, CE is a method of separating an introduced component by a difference in mobility based on a charge, a magnitude, a shape, and the like. The type of the target base sequence (based on the type of the primer) can be identified based on the mobility. Based on the signal of the fluorescent dye, the presence or absence of the target base sequence or the type of a specific base in the target base sequence (based on the type of the substrate incorporated by the single base extension reaction), for example, a wild type and a mutant can be distinguished.

In one embodiment, the step of detecting the wild type and mutant of the target base sequence in the method according to the invention may include a step of quantifying a content ratio of the wild type and the mutant of the target base sequence based on a magnitude of the fluorescence signal. In this case, for example, an abundance ratio of a mutant sequence to a wild type sequence that is required for cancer diagnosis or a frequency of gene mutation can be quantified. In one embodiment, when a plurality of target base sequences to be analyzed include a wild type sequence and a mutant sequence, and a content ratio of the mutant sequence to the wild type sequence is in a range of 0.01% to 10%, for example, in a range of 0.01% to 1%, and further in a range of 0.01% to 0.1%, the target base sequences can be analyzed. In this way, quantitative gene analysis can be performed on the target base sequence.

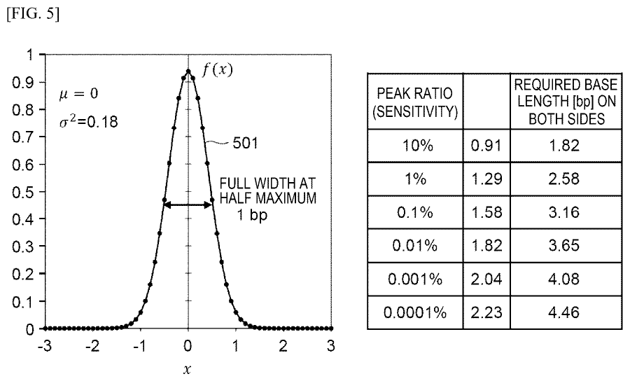

FIG. 5 is a diagram illustrating a percentage of fluorescence intensity peak ratio corresponding to an electrophoretic mobility (a base length) separation when a fluorescence signal of a labeled fluorescent dye that is detected by capillary electrophoresis is assumed to be a Gaussian function. The fluorescence signal of the labeled fluorescent dye that is detected by the capillary electrophoresis is assumed to be a Gaussian function f(x) of the following formula (1).

$$[ Math . 1 ]$$

$$f ( x ) = \frac{1}{\sqrt{2 π} σ} exp \{ - \frac{{( x - μ )}^{2}}{2 {σ}^{2}} \} \qquad ( Formula 1 )$$

In the formula, σ² represents dispersion, and μ represents an average. FIG. 5 illustrates a signal shape 501 when a full width at half maximum FWHM in the following formula (2) is 1 bp.

$$[ Math . 2 ]$$

$$FWHM = 2 \sqrt{2 ln 2} \cdot σ \qquad ( Formula 2 )$$

When considering a peak ratio of the fluorescence intensity, signal values of 10%, 1%, 0.1%, 0.01%, 0.001%, and 0.0001% with respect to a central peak may be detected at base positions with an electrophoretic mobility (a base length) of 0.91 bp, 1.29 bp, 1.58 bp, 1.82 bp, 2.04 bp, and 2.23 bp away from the center, respectively. In reality, it should be noted that the fluorescence signal does not become a Gaussian function itself due to tailing of fluorescent signal, and a half-value width also widens.

In order to quantitatively measure the abundance ratio of the mutant to the wild type, a required separation in base length between primers for adjacent target base sequences, serving as a mobility, can be determined from a relationship table illustrated in FIG. 5. For example, when performing quantitative measurement with a detection sensitivity capable of detecting 1% abundance ratio of the mutant to the wild type, it may be necessary to ensure that a fluorescence signal peak of 1% mutant does not overlap with a fluorescence signal of adjacent targets. Considering that a level at which the signal is not affected is 1/100 of the fluorescence signal for 1% mutation, that is, a peak ratio (a sensitivity) equivalent to 0.01%, then the required separation in base length is 1.82 bp or more. Considering that the Gaussian function spreads on both sides of the graph, with respect to the base length of the primer for the adjacent target base sequence, it can be determined that adjacent primers are preferably designed so as to be separated by 3.65 bp or more, which is twice 1.82 bp. A shape of the fluorescence signal may also change depending on electrophoretic conditions (an injection voltage, an injection time, a separation voltage, a separation time, a temperature, and the like) and specifications of the capillary electrophoresis device itself (theoretically, both are Gaussian functions, but variances and full widths at half maximum may be different).

As described above, the gene analysis may be performed twice or more for the same target base sequence using two or more types of primers. In one embodiment, two or more results (for example, a fluorescence signal and a mass spectrometry signal) obtained for the same target base sequence may be determined separately, or two or more obtained results may be integrated and determined. Since the gene analysis is performed a plurality of times, the accuracy and precision of the gene analysis may be improved by the gene analysis method of the invention.

In one embodiment, the method of the invention may include a step of counting the number of occurrences of a mutant signal obtained by performing the base extension reaction using the two or more types of primers, and determining presence or absence of the mutant with respect to the wild type.

A combination of the single base extension reaction and mass spectrometry, for example, an MassARRAY (registered trademark) system can be adopted as a detection technique. In an embodiment utilizing mass spectrometry, the method of the invention may include: a step of performing a single base extension reaction using a test nucleic acid as a template by using a single base extension reaction primer for detecting the target base sequence; a step of subjecting a product of the single base extension reaction to mass spectrometry; and a step of detecting a wild type and a mutant of the target base sequence in the test nucleic acid based on a difference in size of the product as a difference in signal of the mass spectrometry and information on the wild type and the mutant of the target base sequence as a difference in signal of the mass spectrometry of a base incorporated in the single base extension reaction, wherein the step of performing the single base extension reaction includes performing the single base extension reaction by using two or more types of single base extension reaction primers with different base lengths for a same target base sequence.

The single base extension reaction can be performed in the same manner as described above, except that the substrate (ddNTP) to be incorporated may not have a fluorescent dye. In the mass spectrometry, the type of the incorporated substrate can be identified by a difference in mass, and thus the wild type and the mutant can be distinguished. The substrate may be labeled with a fluorescent dye or the like, and in this case, based on a mass signal of the substrate and a label, the incorporated substrate can be identified, and the wild type or the mutant can be distinguished. As such a method, for example, there has been known an iPLEX (registered trademark) multiplex assay technique using an MassARRAY (registered trademark) system.

The product obtained by the single base extension reaction may be subjected to known mass spectrometry. In the mass spectrometry, by measuring a mass of a product, a product derived from a different primer and a product derived from the wild type or the mutant can be distinguished to provide a mass spectrometry signal. Therefore, by analyzing the mass spectrometry signal, a gene mutation for the same target base sequence can be analyzed.

In one embodiment, the step of detecting the wild type and the mutant of the target base sequence may include a step of quantifying a content ratio of the wild type and the mutant of the target base sequence based on a magnitude of a signal intensity of the mass spectrometry.

Further, a combination of an electrophoresis technique and a multi-base extension reaction, for example, a shifted termination assay (STA) (registered trademark) can be adopted. In an embodiment utilizing multi-base extension reaction, the method of the invention may include: a step of performing a multi-base extension reaction using the test nucleic acid as a template by using a multi-base extension reaction primer for detecting the target base sequence and a multi-base extension reaction substrate having a fluorescent dye; a step of subjecting a product of the multi-base extension reaction to electrophoresis; and a step of detecting a wild type and a mutant of the target base sequence in the test nucleic acid based on a difference in size of the product as a mobility of the electrophoresis and information on the wild type and the mutant of the target base sequence as a mobility of the electrophoresis and a fluorescence signal of the fluorescent dye, wherein the step of performing the multi-base extension reaction includes performing the multi-base extension reaction using two or more types of multi-base extension reaction primers with different base lengths for a same target base sequence.

In the multi-base extension reaction, the test nucleic acid may be used as a template, a primer is bound to a target base sequence, and then the base extension reaction may be performed in the presence of a substrate to which a different fluorescent dye is bound and, optionally, a substrate to which no fluorescent dye is bound. Based on the number (size) of incorporated bases and the type of fluorescent dye, the wild type and the mutant can be distinguished with high specificity. Examples of such a method may include the above-described STA (registered trademark) method.

In one embodiment, the step of detecting the wild type and the mutant of the target base sequence may include a step of quantifying a content ratio of the wild type and the mutant of the target base sequence based on a magnitude of the fluorescence signal.

The invention may not be limited to the above-described base extension reaction, and can also be used to perform genetic analysis of a target base sequence by utilizing an amplification reaction.

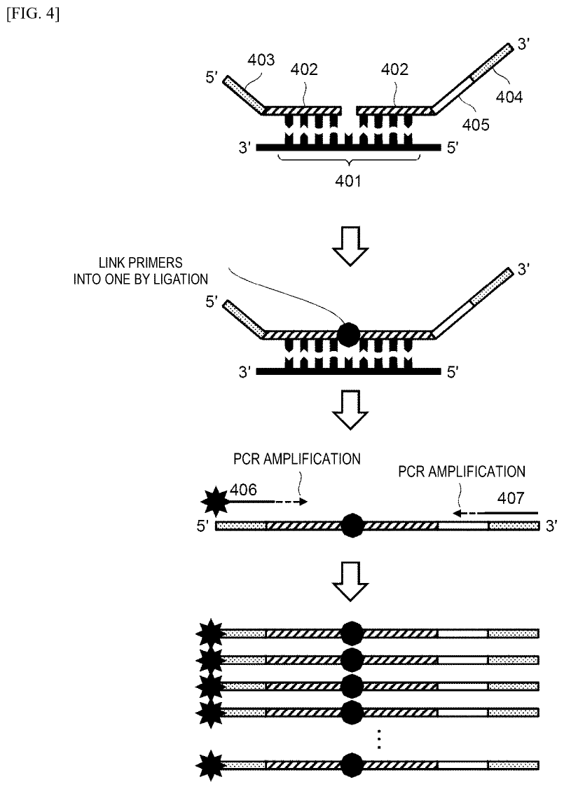

FIG. 4 is a diagram illustrating an embodiment of detection of a target base sequence by an MLPA assay. The invention is applicable not only to SNP typing but also to gene mutation detection using fragment analysis of capillary electrophoresis, and an example thereof will be described. In the MLPA assay, a primer having a base sequence 402 that specifically hybridizes to a target base sequence and a common sequence forward primer 403, and a primer having a base sequence 402 that specifically hybridizes to a target base sequence and a common sequence reverse primer 404 may apply to a target base sequence (a template) 401. At this time, an inserted base sequence 405 for adjusting a base length may be inserted adjacent to the common sequence reverse primer 404. Therefore, a coverage of the target base sequence can be increased by applying a plurality of primers to one type of target base sequence. The two types of primers applying to the target base sequence (the template) 401 may be combined into one by ligation, and the ligated probe may be amplified (for example, by PCR amplification) using a fluorescently labeled forward primer 406 and a reverse primer 407. Since the base length can be adjusted by using the inserted base sequence 405 for adjusting a base length, a plurality of amplification products with different base lengths can be generated for one type of the same target base sequence. Therefore, it can be said that the invention can also be applied to a gene mutation targeted by the MLPA assay. Examples of the gene mutation may include copy number variation (CNV) and epigenetic mutation mainly including methylation. Similarly, the invention is also applicable to a shifted termination assay (STA) (registered trademark) for detecting a gene mutation by a multi-base extension method.

Therefore, in another aspect, the invention relates to a gene analysis method for detecting a target base sequence, wherein the gene analysis method includes: a step of binding, to a test nucleic acid, a forward primer and a reverse primer which specifically hybridize to the target base sequence; a step of performing an amplification reaction using a primer labeled with a fluorescent dye and using, as a template, a ligated probe obtained by ligating the forward primer and the reverse primer; a step of subjecting a product of the amplification reaction to electrophoresis; and a step of detecting a wild type and a mutant of the target base sequence in the test nucleic acid based on a difference in size of the product as a mobility of the electrophoresis and information on the wild type and the mutant of the target base sequence as a fluorescence signal of the fluorescent dye, wherein the reverse primer includes an inserted base sequence for adjusting a base length, thereby allowing two or more types of reverse primers with different base lengths to bind to the test nucleic acid.

The forward primer and the reverse primer that specifically hybridize to the target base sequence can be appropriately designed by those skilled in the art based on the technical common knowledge in the technical field regarding the primer design as described above. In the embodiment, the reverse primer may be provided with an inserted base sequence for adjusting a base length, whereby two or more types of reverse primers with different base lengths are obtained. The inserted base sequence may be contained in the forward primer.

When a target base sequence for which quantitative gene analysis is desired is contained, it is advisable to place a target base sequence item for which higher quantitation is required in a base length region where resolution and accuracy of the capillary electrophoresis device is high (generally, the base length region may be set to around 50 bp to 100 bp, but may vary depending on the device specification). Therefore, in one embodiment, when detecting a plurality of target base sequences, the inserted base sequence contained in the reverse primer for detecting a target base sequence for which quantification is desired is designed to have a base length shorter than that of the inserted base sequence contained in the reverse primer for detecting another target base sequence.

In the embodiment, after the forward primer and the reverse primer are bound to the test nucleic acid, both primers may be ligated. For the ligation, any ligase known in the art can be used, as long as it is an enzyme (a DNA ligase) that can link (ligate) the ends of DNA together via a phosphodiester bond. A representative example of such a DNA ligase may be, but is not limited to, T4 DNA ligase.

The ligated probe obtained by ligation may be used as a template to perform an amplification reaction using a primer labeled with a fluorescent dye. Only one or both of the primers in a pair of primer set may be labeled with a fluorescent dye. The design of the primer and the selection of the fluorescent dye can be appropriately performed by those skilled in the art as described above.

The product obtained by the amplification reaction may be subjected to electrophoresis, and the wild type and the mutant of the target base sequence may be detected in the same manner as described above. In one embodiment, the step of detecting the wild type and the mutant of the target base sequence may include a step of quantifying a content ratio of the wild type and the mutant of the target base sequence based on a magnitude of the fluorescence signal.

As described above, since the gene analysis is performed twice or more for the same target base sequence using two or more types of primers, the accuracy and precision of the gene analysis may be improved.

The gene analysis method according to the invention described above can be easily and quickly performed by a gene analysis device having a necessary configuration or a gene analysis kit including a necessary component.

Therefore, in another aspect, the invention provides a gene analysis device. Such a device includes: a measurement unit configured to perform a single base extension reaction, electrophoresis, and measurement of a fluorescence signal; a data analysis unit including a data processing device and a measurement data storage unit that stores measurement data obtained by the measurement unit; and a control unit, wherein the control unit is configured to decide a number, a type, and a base length of two or more types of primers to be used based on a mutation detection percentage of a target base sequence.

The control unit may further include a reference database that stores previous measurement data. In this case, the control unit may be configured to compare the measurement data stored in the measurement data storage unit with the previous measurement data stored in the reference database to determine a type and a base length of the primer to be used in the single base extension reaction.

The gene analysis device according to the invention may further include an output display unit.

In yet another aspect, the invention provides a gene analysis kit. Such a kit includes: a single base extension reaction primer for detecting a target base sequence; and a single base extension reaction substrate having a fluorescent dye, wherein the single base extension reaction primer includes two or more types of primers with different base lengths for a same target base sequence. Two or more types of primers with different base lengths may be contained for each of a plurality of target base sequences.

The kit according to the invention may contain, in addition to the above components, a buffer constituting a reaction solution, enzymes (polymerase, reverse transcriptase, and the like), a standard sample for calibration, and the like. By providing the primer and the substrate used in the single base extension reaction as the kit, the gene analysis can be performed more quickly and easily.

Hereinafter, a device and a kit for performing a gene analysis method in which a single base extension reaction and electrophoresis are combined will be specifically described.

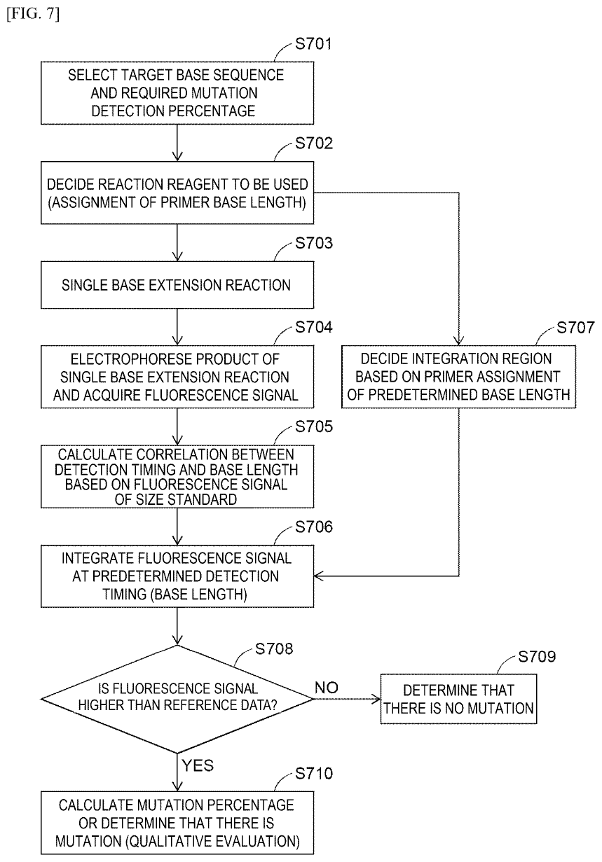

FIG. 7 is a flowchart illustrating an example of a processing procedure in the gene analysis device and the gene analysis kit for implementing the invention. According to the invention, a system can be constructed that can increase the quantitative accuracy of gene mutation detection by freely setting a base length of a primer depending on a target base sequence and a required mutation detection percentage.

First, in step S701, a target base sequence and a required mutation detection percentage may be selected. Here, the “mutation detection percentage” represents an abundance ratio of a mutant to a wild type of the target base sequence to be detected, and based on the mutation detection percentage, a base length of an adjacent primer can be set as illustrated in FIG. 5.

Next, in step S702, a reaction reagent to be used may be selected. Specifically, the base length of the primer that can be used may be assigned as much as possible depending on a detection base length range that is the specification of the analysis device. As described above with reference to FIG. 5, a relationship between a mutation percentage of a detection target and a primer base length may be pre-established, providing guidelines. Subsequently, in step S703, a single base extension reaction may be performed. In step S704, a product of the single base extension reaction may be subjected to electrophoresis, and a fluorescence signal may be obtained. At this time, by mixing a size standard capable of obtaining a fluorescence signal at a predetermined detection position (base length), a relationship between a detection timing of the fluorescence signal during electrophoresis and a base length can be more accurately obtained. In step S705, a step of calculating a correlation between the detection timing and the base length based on a fluorescence signal of the size standard is described. Subsequently, in step S706, the fluorescence signal at the predetermined detection timing (base length) may be integrated. At this time, as illustrated in step S707, an integration region (a base length to be integrated) may be determined from primer assignment of a predetermined base length based on information on the reaction reagent to be used. In step S708, a fluorescence signal of a target may be compared with reference data stored in the reference database or reference data that outputs a baseline value in setting of the reaction reagent to determine whether the fluorescence signal of the target is high. When the fluorescence signal of the target is not higher than the reference data, the target may be determined to be “no mutation” (step S709). When the fluorescence signal of the target is higher than the reference data, the mutation percentage may be calculated or a qualitative evaluation may be made that “there is mutation” in step S710. Since the fluorescence signal is confirmed at a plurality of detection timings by applying a plurality of primers to one type of target base sequence, whether a mutation can be detected can also be determined by counting the number of times that the fluorescence signal is higher than the reference data.

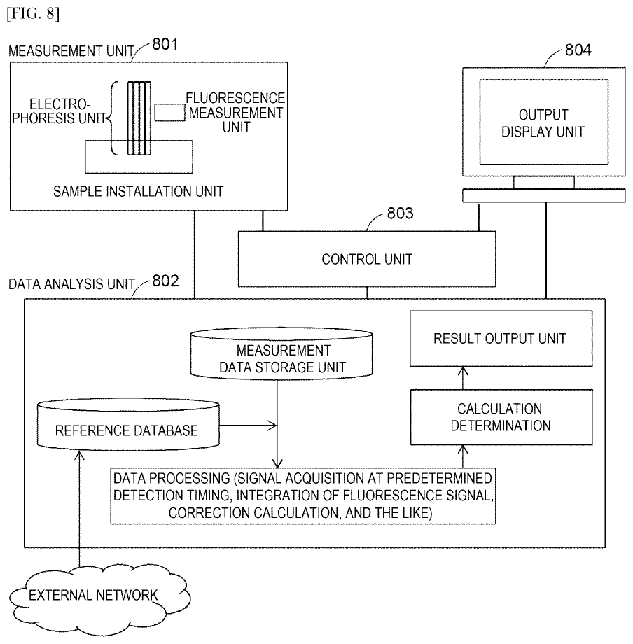

FIG. 8 is a block configuration diagram illustrating an example of functions of the gene analysis device of the invention. Main components of the gene analysis device may be a measurement unit 801, a data analysis unit 802, a control unit 803, and an output display unit 804. In the measurement unit 801, a sample extended by single base may be installed in a sample installation unit, and using capillary electrophoresis, a fluorescence signal over time of the sample flowing through an electrophoresis unit may be measured by a fluorescence measurement unit. The data analysis unit 802 may include a measurement data storage unit for storing measurement data obtained by the measurement unit 801, and a program for executing the data processing can be implemented by software. As illustrated in the flowchart in FIG. 7, contents of the data processing may include acquisition of a fluorescence signal at a predetermined detection timing (a base length), integration of the fluorescence signal of the predetermined base length, correction calculation of a signal drift caused by an increase in electrophoresis time, and the like. At this time, reference data in electrophoresis data stored in the data analysis unit 802 in advance can also be used. Further, the reference data can be updated by transmitting and receiving information to and from an external network.

All functional controls of the measurement unit 801, the data analysis unit 802, and the like can be implemented by software by a processor interpreting and executing a program stored in a memory of the control unit 803. A part or all of the configurations, functional units, processing units, processing methods, and the like described above may be implemented by hardware by, for example, designing with an integrated circuit. Information such as a program, a file, and a database for implementing each function can be stored in a recording device such as a memory, a hard disk, or a solid state drive (SSD), or in a recording medium such as an IC card, an SD card, or a DVD. After the data processing, by calculation determination, it may be determined whether correction of the fluorescence signal using a correction value brings the fluorescence signal within the detectable range of the analysis device, and the result may be output by the output display unit 804. For example, by displaying the integrated fluorescence signal, generally, a user can understand at a glance that the fluorescence signal integrated by applying a plurality of primers is obtained at an S/N ratio higher than that of a signal obtained when a gene mutation is detected by applying one type of primer to one type of target base sequence.

The block configuration diagram illustrated here is an example of a system that is an integrated gene analysis device, and the gene analysis method of the invention can be applied as long as it has the functions of the measurement unit 801, the data analysis unit 802, the control unit 803, and the output display unit 804.

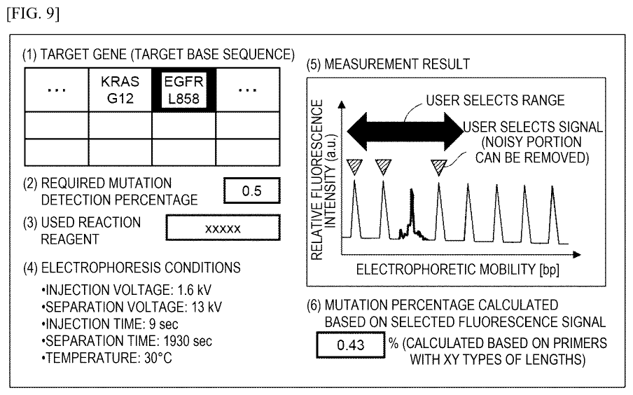

FIG. 9 is a diagram illustrating an example of a user interface screen output by the gene analysis device of the invention. As described above with reference to FIGS. 7 and 8, a target gene (a target base sequence), a required mutation detection percentage, and a reaction reagent to be used corresponding thereto may be input to the gene analysis device. The electrophoresis conditions, such as an injection voltage, an injection time, a separation voltage, a separation time, and a temperature, may be input in the same manner as specifications of a typical capillary electrophoresis device, and measurement results display the electrophoresis conditions as reference data. The measurement results of the electrophoresis may be displayed on a screen, and the user can select a signal derived from a mutation in the target gene (the target base sequence) illustrated in a specific range of an electrophoretic mobility (bp). Accordingly, the settings for the mutation percentage and the method of counting mutation detection can be changed. For example, when the accuracy is poor in a long base length portion of a primer, or when characteristics of the reaction reagent cause specific noise to be detected in the base length portion, the user can selectively exclude the signal. The result of calculating the mutation percentage based on the selected fluorescence signal may be displayed on the screen.

The invention will be described in detail with reference to the following Example, but the Example is merely provided for describing the invention, and does not limit the scope of the invention disclosed in the present application.

### Example

An OncoSpan DNA Reference Standard (Horizon) was used as a standard sample containing a gene mutation, and EGFR L858, which is a type of cancer driver gene, was used as a target gene. The EGFR L858 mutation is a sequence EGFR L858R in which leucine (L: CUG) at position 858 is substituted with arginine (R: CGG) by one base. First, PCR for cloning was performed using the standard samples containing EGFR L858 wild type (EGFR L858WT) and mutant (L858R) genes as a template. The PCR product was transformed into Escherichia coli and cultured in LB medium, and then amplified by colony direct PCR. A sequencing reaction was performed using a BigDye Terminator Sequencing Kit (Thermo Fisher Scientific Inc.), and after purification, the sequence was confirmed using a genetic analyzer SeqStudio, and then the plasmid was extracted. The extracted plasmid was used as a template for PCR, and the amplified product was used as a target gene.

For a target base sequence (a template) in the target gene, 11 types of EGFR L858 primers (Table 1) with base lengths between 50 bp and 100 bp and differing by 5 bp each were mixed at 0.2 μM each, along with 1 U of DNA polymerase and fluorescent dye-modified ddNTPs (T-labeled: ROX-ddUTP, G-labeled: R110-ddGTP) (PerkinElmer), and a single base extension reaction was performed in a thermal cycler under conditions of [96° C.×10 sec->50° C.×5 sec→60° C.×30 sec]×25 cycles. A concentration of the target template DNA was such that a wild type was fixed at 100 fmol, and a mutant was varied as follows: 0 fmol (corresponding to 0% mutation), 0.1 fmol (corresponding to 0.1% mutation), 0.3 fmol (corresponding to 0.3% mutation), 1 fmol (corresponding to 1% mutation), 3 fmol (corresponding to 3% mutation), and 10 fmol (corresponding to 10% mutation). In the primers illustrated in Table 1, underlined portions are target base sequences, and other portions are universal sequences.

TABLE 1

| Primer | | Base | SEQ ID |
|---|---|---|---|
| name | Sequence (5′→3′) | length | NO: |
| #1 | ATGGGTGGACGTGACACTAT | 50 | 1 |
| | AGCAGCATGTCAAGATCACA | | |
| | GATTTTGGGC | | |
| #2 | ATGGGTGGACTTTAGGTGAC | 55 | 2 |
| | ACTATAGCAGCATGTCAAGA | | |
| | TCACAGATTTTGGGC | | |
| #3 | ATGGGTGGACAGCTATTTAG | 60 | 3 |
| | GTGACACTATAGCAGCATGT | | |
| | CAAGATCACAGATTTTGGGC | | |
| #4 | ATGGGTGGACAGTCAAGCTA | 65 | 4 |
| | TTTAGGTGACACTATAGCAG | | |
| | CATGTCAAGATCACAGATTT | | |
| | TGGGC | | |
| #5 | ATGGGTGGACCGGCCAGTCA | 70 | 5 |
| | AGCTATTTAGGTGACACTAT | | |
| | AGCAGCATGTCAAGATCACA | | |
| | GATTTTGGGC | | |
| #6 | ATGGGTGGACAACGACGGCC | 75 | 6 |
| | AGTCAAGCTATTTAGGTGAC | | |
| | ACTATAGCAGCATGTCAAGA | | |
| | TCACAGATTTTGGGC | | |
| #7 | ATGGGTGGACCGTAAAACGA | 80 | 7 |
| | CGGCCAGTCAAGCTATTTAG | | |
| | GTGACACTATAGCAGCATGT | | |
| | CAAGATCACAGATTTTGGGC | | |
| #8 | ATGGGTGGACTATGACGTAA | 85 | 8 |
| | AACGACGGCCAGTCAAGCTA | | |
| | TTTAGGTGACACTATAGCAG | | |
| | CATGTCAAGATCACAGATTT | | |
| | TGGGC | | |
| #9 | ATGGGTGGACACAGCTATGA | 90 | 9 |
| | CGTAAAACGACGGCCAGTCA | | |
| | AGCTATTTAGGTGACACTAT | | |
| | AGCAGCATGTCAAGATCACA | | |
| | GATTTTGGGC | | |
| #10 | ATGGGTGGACAGGAAACAGC | 95 | 10 |
| | TATGACGTAAAACGACGGCC | | |
| | AGTCAAGCTATTTAGGTGAC | | |
| | ACTATAGCAGCATGTCAAGA | | |
| | TCACAGATTTTGGGC | | |
| #11 | ATGGGTGGACTCTCCAGGAA | 100 | 11 |
| | ACAGCTATGACGTAAAACGA | | |
| | CGGCCAGTCAAGCTATTTAG | | |
| | GTGACACTATAGCAGCATGT | | |
| | CAAGATCACAGATTTTGGGC | | |

After the single base extension reaction, a dephosphorylation reaction (SAP) treatment was performed to prevent interference with the fluorescent labeled ddNTPs, which is an unreacted substrate. To 10 μL of the reaction product, 1 μL of SAP was added, and the mixture was reacted at 37° C. for 1 hour, and then at 75° C. for 15 minutes. The SAP-treated sample was mixed with a size marker and Hi-Di Formamide, and the mixture was heat-treated at 95° C. for 5 minutes, after which fragment analysis was performed using a CE sequencer DS3000 (Hitachi High-Tech Corporation).

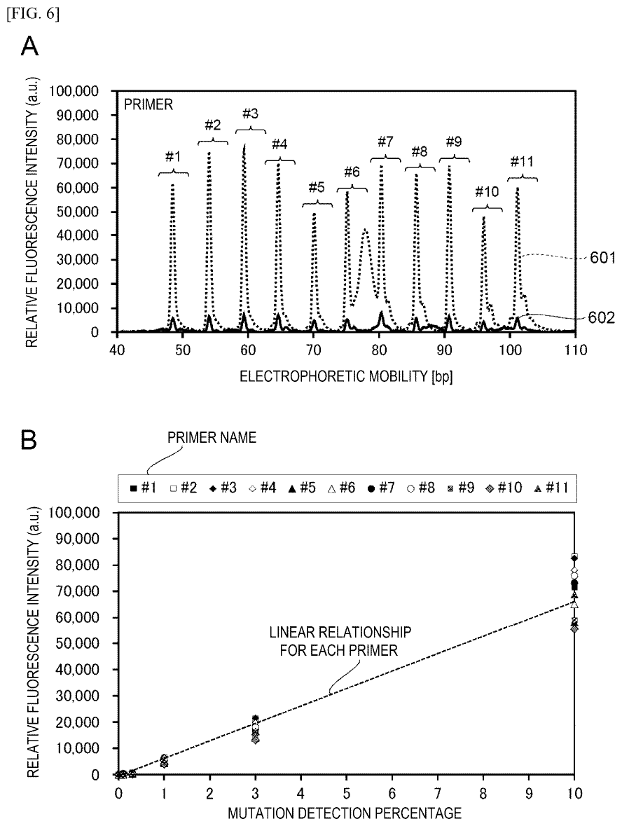

FIG. 6, A and B are diagrams illustrating results of the fragment analysis in which the single base extension reaction was performed using 11 types of primers with different base lengths (Table 1) with the gene mutation EGFR L858R targeted. Generally, mutation quantification is performed for a plurality of gene items by assigning each type of primers with different base lengths to each type of target in one round of electrophoresis, but in the present Example, 11 types of primers with different base lengths (primers #1, 2, 3, . . . , 11) are assigned to one type of target (EGFR L858R), fluorescence signals are detected at 11 different electrophoretic mobility (bp) positions. As illustrated in FIG. 6, A, when a ratio of the mutant to the wild type is 1%, a mutant signal 602 corresponding to 1% mutation is detected with respect to a wild type signal 601. By detecting 11 positions, the target base sequence in the target gene can be quantified with a coverage of 11 times in one round of electrophoresis.

The present Example was performed with the number of experiments N=4. From peaks of relative fluorescence intensity obtained by changing an abundance ratio of the mutant (a mutation detection percentage) to 0%, 0.1%, 0.3%, . . . , a linear relationship can be found between the abundance ratio of the mutant and the relative fluorescence intensity (FIG. 6, B). In this case, when focusing on only one type of primer, a gene mutation detection lower limit (3SD; a mutation percentage equivalent to three times an SD value when the mutation percentage was 0%) was 0.066%=0.046% on average, and varied between 0.033% and 0.205% for the 11 types of primers. This is because when a primer with one specific base length is used, mutations can be detected at 0.033% or only at 0.205%, and thus the reliability (accuracy) of gene mutation detection is low. On the other hand, when the 11 types of primers with different base lengths were used, the gene mutation detection lower limit was 0.050%+0.001% (N=4), with very small variation and sufficient stability in terms of reliability (accuracy) of gene mutation detection.

At this time, in order to increase the S/N ratio, fluorescence signals derived from 11 types of primers with different base lengths may be added. In order to detect low-frequency and low-level gene mutations, the ability to increase the S/N ratio in one round of electrophoresis and perform high-sensitivity detection is an extremely useful feature, for example, when used in liquid biopsy, where sample amounts are limited. In the present Example, 11 types of primers with base lengths between 50 bp and 100 bp, differing by 5 bp, were used, but a base length to which fragment analysis using a capillary electrophoresis device can be applied is 100 bp or more. Therefore, when an electrophoretic mobility range is expanded, primers with more different base lengths can be used, and it is believed that the data reliability in quantitative analysis of gene mutations can be further improved.

For low-level gene mutations (for example, an abundance ratio of less than 0.1%), using a plurality of primers with different base lengths may result in a fluorescence signal having a certain value being detected in some cases but not in others. In this case, the number of times the fluorescence signal from the mutant exceeds a certain value can be counted to determine the presence or absence of the mutant for the wild type. In particular, in a test for early detection such as cancer diagnosis of liquid biopsy, determination of the presence or absence of a gene mutation is important, and therefore the invention is effective not only in quantifying allele frequency but also in determining the presence or absence of the gene mutation.

The fluorescent dyes used in the above Example were x-rhodamine (ROX) and rhodamine 110 (R110). However, the fluorescent dyes that can be used in the invention are not limited thereto, and any fluorescent dye that is generally used to label a nucleic acid probe may be used. Other than rhodamine derivatives such as rhodamine 6G (R6G) and tetramethylrhodamine (TAMRA), for example, fluorescein or fluorescein isothiocyanate (FITC), which is an derivative thereof, Alexa 488, Alexa 532, cy3, cy5, and Texas can be used. The fluorescent dye can be freely determined according to an excitation wavelength of laser light mounted on a capillary electrophoresis device to be used.

The invention is not limited to the embodiments described above and includes various modifications. For example, the above embodiments have been described in detail to facilitate understanding of the invention, and the invention is not necessarily limited to those including all the configurations described above. A part of a configuration of a certain embodiment can be replaced with a configuration of another embodiment, and a configuration of another embodiment can be added to a configuration of a certain embodiment. It is possible to add, delete, or replace a part of configurations of each embodiment with other configurations.

### REFERENCE SIGNS LIST

101: target base sequence (template)
102: primer with different base length for each target
103: fluorescent dye
104: product of single base extension reaction
201: primer with short-adjusted base length
202: primer with long-adjusted base length
203: product from primer with short-designed base length
204: product from primer with long-adjusted base length
301: target base sequence (template)
302: complementary sequence (template antisense) of target base sequence
303: primer with adjusted base length (forward primer)
304: complementary strand primer with adjusted base length (reverse primer)
401: target base sequence (template)
402: base sequence specifically hybridizing to target base sequence
403: common sequence forward primer
404: common sequence reverse primer
405: inserted base sequence for adjusting base length
406: fluorescently labeled forward primer
407: reverse primer
501: signal shape when full width at half maximum is 1 bp
601: wild type signal
602: mutant signal
801: measurement unit
802: data analysis unit
803: control unit
804: output display unit

### [Sequence Table Free Text]

SEQ ID NOs: 1 to 11: DNA (synthetic oligonucleotides)

1. A gene analysis method for detecting a target base sequence, the gene analysis method comprising:
a step of performing a base extension reaction using a test nucleic acid as a template by using a primer specific for the target base sequence;
a step of subjecting a product of the reaction to electrophoresis or mass spectrometry; and
a step of detecting a wild type and a mutant of the target base sequence in the test nucleic acid based on a difference in size of the product determined by the electrophoresis or the mass spectrometry and information on the wild type and the mutant of the target base sequence, wherein
the step of performing the base extension reaction comprises performing the base extension reaction using two or more types of primers with different base lengths for a same target base sequence.

2. The method according to claim 1, further comprising:
a step of counting the number of occurrences of a mutant signal obtained by performing the base extension reaction using the two or more types of primers, and determining presence or absence of the mutant with respect to the wild type.

3. The method according to claim 1, comprising:
a step of performing a single base extension reaction using a test nucleic acid as a template by using a single base extension reaction primer for detecting the target base sequence and a single base extension reaction substrate having a fluorescent dye;
a step of subjecting a product of the single base extension reaction to electrophoresis; and
a step of detecting a wild type and a mutant of the target base sequence in the test nucleic acid based on a difference in size of the product as a mobility of the electrophoresis and information on the wild type and the mutant of the target base sequence as a fluorescence signal of the fluorescent dye, wherein
the step of performing the single base extension reaction comprises performing the single base extension reaction using two or more types of single base extension reaction primers with different base lengths for a same target base sequence.

4. The method according to claim 3, wherein
the step of detecting the wild type and the mutant of the target base sequence comprises a step of quantifying a content ratio of the wild type and the mutant of the target base sequence based on a magnitude of the fluorescence signal.

5. The method according to claim 3, wherein
the two or more types of single base extension reaction primers comprise primers having a same orientation or primers having different orientations.

6. The method according to claim 3, wherein
when detecting a plurality of target base sequences, a single base extension reaction primer for detecting a target base sequence for which quantification is desired is designed to have a base length shorter than that of the single base extension reaction primer for detecting another target base sequence.

7. The method according to claim 1, comprising:
a step of performing a single base extension reaction using a test nucleic acid as a template by using a single base extension reaction primer for detecting the target base sequence;
a step of subjecting a product of the single base extension reaction to mass spectrometry; and
a step of detecting a wild type and a mutant of the target base sequence in the test nucleic acid based on a difference in size of the product as a difference in signal of the mass spectrometry and information on the wild type and the mutant of the target base sequence as a difference in signal of the mass spectrometry of a base incorporated in the single base extension reaction, wherein
the step of performing the single base extension reaction comprises performing the single base extension reaction by using two or more types of single base extension reaction primers with different base lengths for a same target base sequence.

8. The method according to claim 7, wherein
the step of detecting the wild type and the mutant of the target base sequence comprises a step of quantifying a content ratio of the wild type and the mutant of the target base sequence based on a magnitude of a signal intensity of the mass spectrometry.

9. The method according to claim 1, comprising:
a step of performing a multi-base extension reaction using the test nucleic acid as a template by using a multi-base extension reaction primer for detecting the target base sequence and a multi-base extension reaction substrate having a fluorescent dye;
a step of subjecting a product of the multi-base extension reaction to electrophoresis; and
a step of detecting a wild type and a mutant of the target base sequence in the test nucleic acid based on a difference in size of the product as a mobility of the electrophoresis and information on the wild type and the mutant of the target base sequence as a mobility of the electrophoresis and a fluorescence signal of the fluorescent dye, wherein
the step of performing the multi-base extension reaction comprises performing the multi-base extension reaction using two or more types of multi-base extension reaction primers with different base lengths for a same target base sequence.

10. The method according to claim 9, wherein
the step of detecting the wild type and the mutant of the target base sequence comprises a step of quantifying a content ratio of the wild type and the mutant of the target base sequence based on a magnitude of the fluorescence signal.

11. A gene analysis method for detecting a target base sequence, the gene analysis method comprising:
a step of binding, to a test nucleic acid, a forward primer and a reverse primer which specifically hybridize to the target base sequence;
a step of performing an amplification reaction using a primer labeled with a fluorescent dye and using, as a template, a ligated probe obtained by ligating the forward primer and the reverse primer;
a step of subjecting a product of the amplification reaction to electrophoresis; and
a step of detecting a wild type and a mutant of the target base sequence in the test nucleic acid based on a difference in size of the product as a mobility of the electrophoresis and information on the wild type and the mutant of the target base sequence as a fluorescence signal of the fluorescent dye, wherein
the reverse primer comprises an inserted base sequence for adjusting a base length, thereby allowing two or more types of reverse primers with different base lengths to bind to the test nucleic acid.

12. The method according to claim 11, wherein
the step of detecting the wild type and the mutant of the target base sequence comprises a step of quantifying a content ratio of the wild type and the mutant of the target base sequence based on a magnitude of the fluorescence signal.

13. The method according to claim 11, wherein
when detecting a plurality of target base sequences, the inserted base sequence contained in the reverse primer for detecting a target base sequence for which quantification is desired is designed to have a base length shorter than a base length of the inserted base sequence contained in the reverse primer for detecting another target base sequence.

14. A gene analysis device for implementing the method according to claim 3, the gene analysis device comprising:
a measurement unit configured to perform a single base extension reaction, electrophoresis, and measurement of a fluorescence signal;
a data analysis unit comprising a data processing device and a measurement data storage unit that stores measurement data obtained by the measurement unit; and
a control unit, wherein
the control unit is configured to decide a number, a type, and a base length of two or more types of primers to be used based on a mutation detection percentage of a target base sequence.

15. A gene analysis kit for implementing the method according to claim 3, the gene analysis kit comprising:
a single base extension reaction primer for detecting a target base sequence; and
a single base extension reaction substrate having a fluorescent dye, wherein
the single base extension reaction primer comprises two or more types of primers with different base lengths for a same target base sequence.
